(19) United States
(12) Plant Patent Application Publication
(10) Pub. No.: US 2002/0170097 P1
(43) Pub. Date: Nov. 14, 2002

(54) Climbing rose plant named 'Meidrason'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 10/114,987

(22) Filed: Apr. 4, 2002

(30) Foreign Application Priority Data
Apr. 4, 2001 (FR) 17668

(51) Int. Cl. A01H 5/00

(52) U.S. Cl. PLT/115

(57) ABSTRACT

A new and distinct variety of Climbing rose plant is provided which abundantly forms very double dark red blossoms having a strong fragrance that are of the old rose type. The stability of the flower coloration is good. The plant displays good vigor and exceptional resistance to diseases. The foliage is very dense, medium green and semi-glossy, and contrasts nicely with the blossom coloration. The new variety is particularly well suited for growing as attractive ornamentation in the landscape where a climbing rose plant is desired.

### SUMMARY OF THE INVENTION

The new variety of Rosa hybrida Climbing rose plant was created by artificial pollination wherein two parents were crossed which previously had been studied in the hope that they would contribute the desired characteristics. The female parent (i.e., the seed parent) was the product of the ‘Delsap’ variety (non-patented in the United States). The male parent (i.e., the pollen parent) was the ‘Kortello’ variety (non-patented in the United States). The parentage of the new variety can be summarized as follows:

‘Delsap’×‘Kortello’.

The seeds resulting from the above pollination were sown and small plants were obtained which were physically and biologically different from each other. Selective study resulted in the identification of a single plant of the new variety.

It was found that the new rose variety of the present invention:

(a) exhibits a climbing growth habit,

(b) abundantly forms attractive very double dark red blossoms having a strong fragrance that are of the old rose type,

(c) forms very dense medium green semi-glossy foliage that contrasts well with the blossom coloration,

(d) displays good vigor,

(e) displays exceptional disease resistance, and

(f) is particularly well suited for growing as attractive ornamentation in the landscape.

The stability of the flower coloration is good.

The new variety well meets the needs of the horticultural industry and can be used to advantage for growing in the landscape where an attractive climbing rose plant is desired.

The new variety of the present invention can be readily distinguished from its ancestors. For instance, the ‘Delsap’ variety displays an upright growth habit, and ‘Kortello’ variety displays a shrub growth habit and forms blossoms having only approximately 35 petals.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meidrason’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were approximately one year of age and were observed during June while budded on Rosa laxa understock and growing outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower plan view—obverse;

FIG. 7—illustrates a specimen of an open flower plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a specimen of a leaf with seven leaflets—plan view—upper surface.

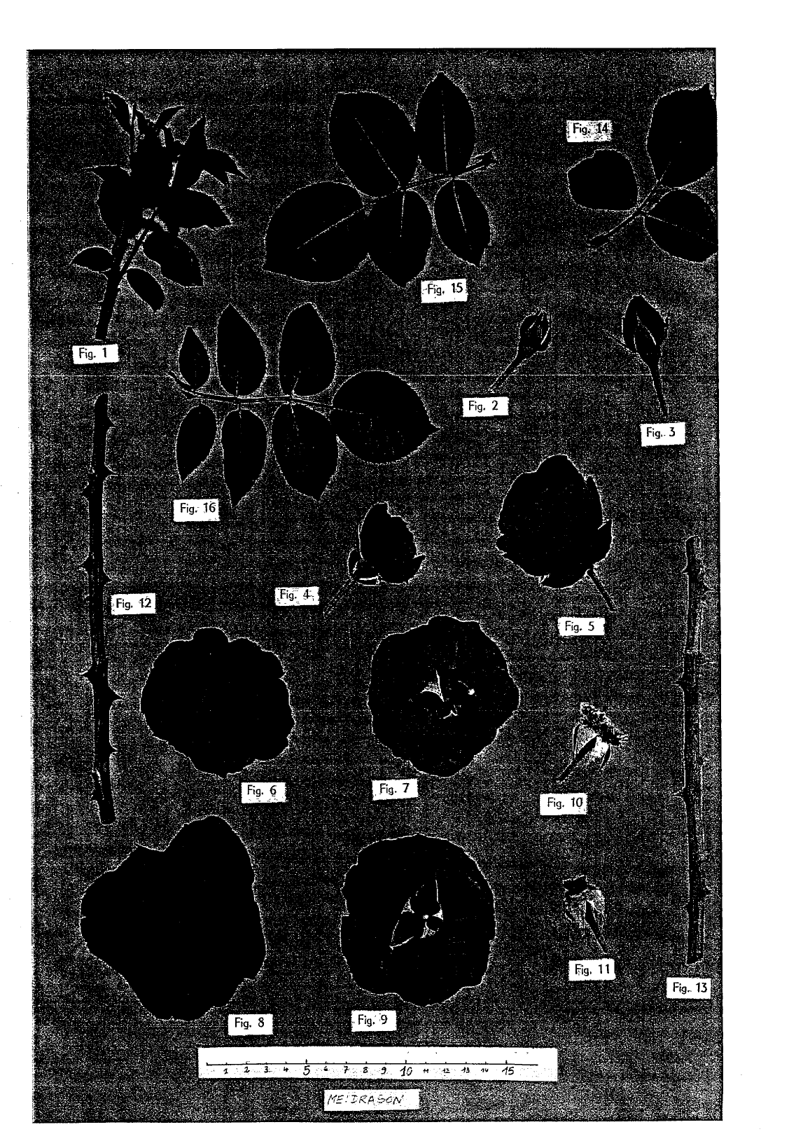

### DETAILED DESCRIPTION

The chart used in the identification of the colors is that of The Royal Horticultural Society (R.H.S. Colour Chart). The description is based on the observation of one year-old plants during June while budded on Rosa laxa understock and growing outdoors at Le Cannet des Maures, Var, France.

Class: Climbing.

Plant:

Height.—approximately 170 to 200 cm on average at the end of the growing season.

Habit.—climbs well with support.

Branches:

Color.—young stems: medium green with some anthocyanin coloration. adult wood: medium green. thorns size: variable (as illustrated). quantity: fairly numerous (as illustrated). configuration: fairly straight on upper surface and concave on the under surface (as illustrated).

Leaves:

Stipules.—adnate, pectinate, and rather broad.

Petioles.—upper surface: non-glandular. under surface: smooth.

Leaflets.—number: 3, 5, and 7 (most often). shape: somewhat ovate with a rounded base and an acute tip. serration: simple and fairly regular (as illustrated). texture: smooth and fairly thick. general appearance: very dense, and medium green with a semi-glossy aspect. color (young foliage): upper surface: medium green with some anthocyanin coloration. under surface: medium green with some anthocyanin coloration. color (adult foliage): upper surface: medium green. under surface: medium green and lighter than the upper surface.

Inflorescence

Number of flowers.—commonly in clusters of 2 to 5 flowers per stem.

Sepals.—tomentose on the upper surface, elongated, and commonly with small extensions (as illustrated).

Buds.—shape: ovoid. size: medium (as illustrated). length: approximately 2 cm on average. color: upper surface: Cardinal Red, Red Group 53B. under surface: Cardinal Red, Red Group 53C.

Flower.—shape: umbrella-shaped when fully open and of the old rose type. diameter: approximately 10 to 11 cm on average. color (when blooming): upper surface: Cardinal Red, Red Group 53B. under surface: Cardinal Red, Red Group 53C. fragrance: strong. petal number: approximately 95 to 100 on average under normal growing conditions. petal arrangement: verticillate. petal drop: good with the petals commonly detaching cleanly before drying. anthers: arranged regularly around the styles. receptacle: funnel-shaped in longitudinal section.

Development

Vegetation.—very strong.

blooming.—very abundant and nearly continuous.

Resistance to diseases.—excellent.

I claim:

1. A new and distinct variety of rose plant characterized by the following combination of characteristics:
(a) exhibits a climbing growth habit,
(b) abundantly forms attractive very double dark red blossoms having a strong fragrance that are of the old rose type,
(c) forms very dense medium green semi-glossy foliage that contrasts well with the blossom coloration,
(d) displays good vigor,
(e) displays exceptional disease resistance, and
(f) is particularly well suited for growing as attractive ornamentation in the landscape; substantially as herein shown and described.
